(19) United States
(12) Patent Application Publication
(10) Pub. No.: US 2012/0016355 A1
(43) Pub. Date: Jan. 19, 2012

(54) SYSTEM AND METHOD FOR REGULATING COOLANT FLOW THROUGH A CATHETER AND AN EXPANSION ELEMENT OF A CRYOABLATION SYSTEM

(75) Inventors: Leonard J. George, Scotts Valley, CA (US); David T. Lawrence, Saratoga, CA (US); James David Harney, San Diego, CA (US)

(73) Assignee: Boston Scientific SciMed, Inc., Maple Grove, MN (US)

(21) Appl. No.: 13/176,533

(22) Filed: Jul. 5, 2011

Related U.S. Application Data
(60) Provisional application No. 61/364,091, filed on Jul. 14, 2010.

(51) Int. Cl. A61B 18/02

(52) U.S. Cl. 606/21

(57) ABSTRACT

A cryoablation system includes a catheter for insertion into patient vasculature. The catheter includes a coolant transfer tube configured to receive and transfer coolant from a coolant source to an expansion element coupled to a distal portion of the catheter. The expansion element is in fluid communication with the coolant transfer tube and at least one coolant outtake region within the catheter. The coolant outtake region is configured and arranged to receive and transfer coolant from the expansion element to a reduced pressure region. A coolant-flow regulation system is at least partially disposed in the catheter. The coolant-flow regulation system is configured and arranged for regulating a rate of coolant flow within the cryoablation system by providing an adjustable pressure differential along the at least one coolant outtake region. A control module is coupled to the catheter. The control module includes a processor for controlling the coolant-flow regulation system.

### CROSS-REFERENCE TO RELATED APPLICATIONS

This application claims the benefit under 35 U.S.C. §119(e) of U.S. Provisional Patent Application Ser. No. 61/364,091 filed on Jul. 14, 2010, which is incorporated herein by reference.

### TECHNICAL FIELD

The present invention is directed to the area of cryoablation systems and methods of making and using the systems. The present invention is also directed to systems and methods of making and using cryoablation systems that include a catheter, a distally-disposed expansion element, and a coolant-flow regulation system, as well as methods of making and using the catheter, expansion element, and coolant-flow regulation system.

### BACKGROUND

Cryoablation systems have been used to reduce, or even eliminate, undesired electrical activity between adjacent cardiac tissues of the heart (arrhythmias). One common type of arrhythmia, atrial fibrillation, is a result of abnormal electrical signals interfering with the normal electrical signal propagation along the tissues of the heart. Atrial fibrillation often originates near the ostia of the pulmonary veins. Cryoablation systems can be used to form lesions on patient tissue in proximity to the ostia, where the pulmonary veins open into the left atrium of the heart. The cold-induced lesions can effectively block the initiation or propagation of the abnormal electrical signals, thereby preventing the abnormal electrical signals from interfering with the normal electrical signal propagation along the tissues of the heart.

### BRIEF SUMMARY

In one embodiment, a cryoablation system includes a catheter having a distal portion, a proximal portion, and a length. The catheter is configured and arranged for insertion into patient vasculature. The catheter includes a coolant transfer tube extending along the catheter. The coolant transfer tube defines a lumen configured and arranged to receive and transfer coolant from a coolant source. At least one coolant outtake region extends along the catheter. An expansion element is coupled to the distal portion of the catheter. The expansion element is in fluid communication with the coolant transfer tube and the at least one coolant outtake region. The coolant outtake region is configured and arranged to receive and transfer coolant from the expansion element to a reduced pressure region. A coolant-flow regulation system is at least partially disposed in the catheter. The coolant-flow regulation system is configured and arranged for regulating a rate of coolant flow within the cryoablation system by providing an adjustable pressure differential along the at least one coolant outtake region. A control module is coupled to the catheter. The control module includes a processor for controlling the coolant-flow regulation system.

In another embodiment, a method for cryoablating patient tissue includes inserting a catheter and an expansion element in patient vasculature, the catheter having a distal portion, a proximal portion, and a length. The catheter includes a coolant transfer tube extending along the catheter and at least one coolant outtake region. The expansion element is disposed at the distal portion of the catheter. The expansion element is guided in proximity to a target ablation location within the patient. Coolant is drawn from a coolant source such that coolant flows along the coolant transfer tube and enters into the expansion element, thereby reducing the temperature of the expansion element to a temperature sufficiently low enough to ablate patient tissue at the target ablation location upon contact. A pressure differential is adjusted along the at least one coolant outtake region using a coolant-flow regulation system at least partially disposed in the catheter. A rate of coolant flow into the coolant transfer tube is adjusted in response to the adjustment of the pressure differential along the at least one coolant outtake region. Patient tissue is contacted at the target ablation location with the expansion element for a time period adequate to ablate tissue contacting the expansion element.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic partial cross-sectional and partial block diagram view of one embodiment of a cryoablation system, according to the invention;

FIG. 2A is a schematic longitudinal cross-sectional view of one embodiment of an expansion element coupled to a distal portion of a catheter of the cryoablation system of FIG. 1, the expansion element in a deflated configuration, according to the invention;

FIG. 2B is a schematic longitudinal cross-sectional view of one embodiment of an expansion element coupled to a distal portion of a catheter of the cryoablation system of FIG. 1, the expansion element an inflated configuration, according to the invention;

FIG. 3 is a schematic longitudinal cross-sectional view of one embodiment of a distal portion of the catheter of FIG. 2A disposed in a sheath, according to the invention; and

FIG. 4 is a schematic view of one embodiment of a coolant-flow regulation system for regulating exhaust pressure differential within the cryoablation system of FIG. 1, according to the invention.

### DETAILED DESCRIPTION

The present invention is directed to the area of cryoablation systems and methods of making and using the systems. The present invention is also directed to systems and methods of making and using cryoablation systems that include a catheter, a distally-disposed expansion element, and a coolant-flow regulation system, as well as methods of making and using the catheter, expansion element, and coolant-flow regulation system.

A cryoablation system can include a catheter configured and arranged for transporting coolant to and from a target location within a patient, an expansion element disposed at a distal portion of the catheter for ablating contacted patient tissue, a coolant source coupled to the catheter for supplying the coolant, a control module for controlling or monitoring one or more of the operations of the system, and a coolant-flow regulation system. The expansion element can be positioned at a target location in patient vasculature (e.g., the left atrium of the heart) and the coolant can be input to the catheter and directed to the expansion element. When the coolant contacts the expansion element, the coolant expands and absorbs heat, thereby causing the expansion element to reduce in temperature to a level low enough to ablate patient tissue upon contact. As the coolant flow ceases, the coolant flows out of the expansion element and back to a proximal end of the catheter. The catheter may then be removed from patient vasculature. In at least some embodiments, when the coolant flows out of the expansion element, the expansion element deflates and the catheter may be removed from patient vasculature.

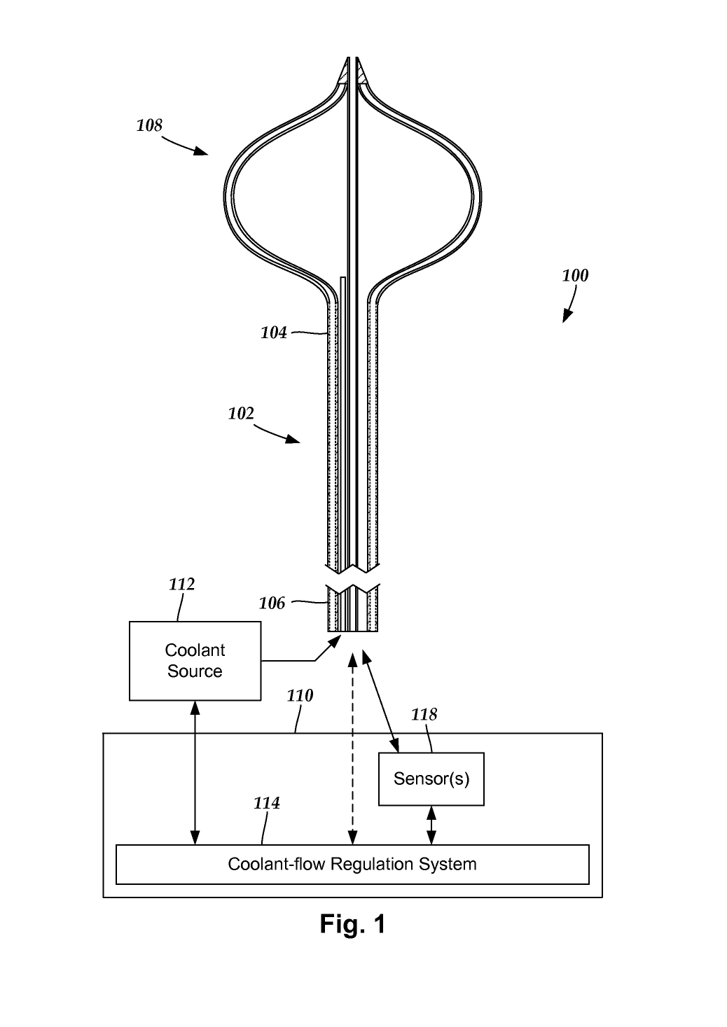

FIG. 1 illustrates schematically one embodiment of a cryoablation system 100. The cryoablation system 100 includes a catheter 102 with a distal portion 104 and a proximal portion 106. An expansion element 108 is coupled to the distal portion 104 of the catheter 102. A control module 110, a coolant source 112, and a coolant-flow regulation system 114 are each coupled to the catheter 102. The coolant-flow regulation system 114 regulates the flow of coolant within the catheter 102. In at least some embodiments, the coolant-flow regulation system 114 also regulates pressure within the expansion element 108.

In at least some embodiments, the coolant-flow regulation system 114 regulates the rate of coolant flow within the catheter 102 by controlling the output of coolant from the expansion element 108. In at least some embodiments, the coolant-flow regulation system 114 also regulates the pressure within the expansion element 108 by controlling the output of coolant from the expansion element 108. In at least some embodiments, the coolant-flow regulation system 114 regulates the rate of coolant flow within the catheter 102 such that the pressure remains constant in the expansion element 108 during a cryoablation procedure. Accordingly, in at least some embodiments, the coolant-flow regulation system 114 regulates the rate of coolant flow within the catheter 102 such that the boiling point temperature of the coolant remains constant in the expansion element during a cryoablation procedure.

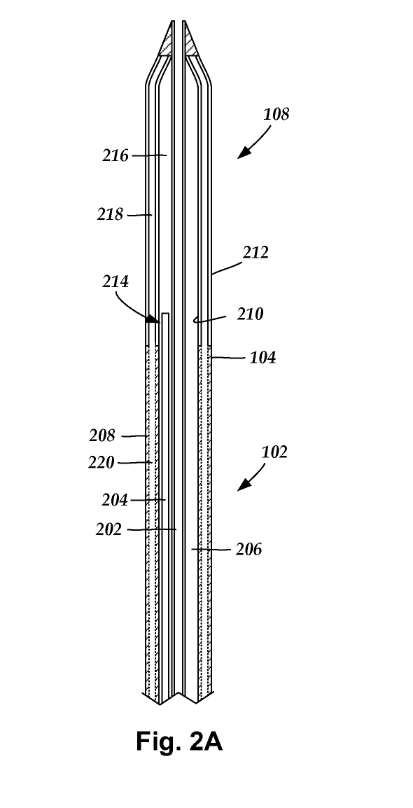

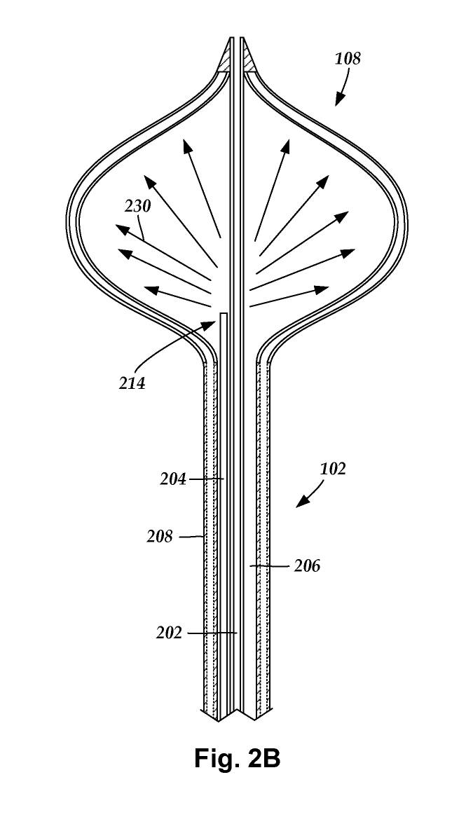

In at least some embodiments, the coolant-flow regulation system 114 regulates the rate of coolant flow within the catheter 102 or pressure within the expansion element 108 by varying pressure within the at least one coolant outtake region (206 in FIG. 2). In at least some embodiments, the coolant-flow regulation system 114 includes an adjustable vacuum source. In at least some embodiments, the coolant-flow regulation system 114 includes one or more adjustable valves open to an external environment. In at least some embodiments, the coolant-flow regulation system 114 also includes one or more sensors 118 for monitoring one or more conditions (e.g., pressure, temperature, or the like) within at least one of the catheter 102 or the expansion element 108.

In at least some embodiments, the coolant source 112 includes a coolant under pressure. Many different coolants may be used to provide a low enough temperature to ablate tissue upon contact. In preferred embodiments, the coolant is a low freezing point liquid with a low vaporization temperature which may be input to the catheter 102 as a liquid that is sprayed into the expansion element 108, where the liquid coolant absorbs heat and is vaporized and atomized. Examples of suitable liquids include, for example, a liquefied gas (e.g., nitrogen, nitrous oxide, carbon dioxide, or the like), one or more chlorofluorocarbons, one or more hydrochlorofluorocarbons, ethanol mixtures, saline solutions, or the like. It will be understood that a combination of one or more coolants may also be used in the cryoablation system 100.

During a typical cryoablation procedure, the distal portion 104 of the catheter 102 is inserted into patient vasculature for delivery of the expansion element 108 to an ablation site. As discussed above, in at least some embodiments the expansion of the coolant causes a corresponding expansion of the expansion element. FIG. 2A is a schematic longitudinal cross-sectional view of one embodiment of the distal portion 104 of the catheter 102 and the expansion element 108. In FIG. 2A, the expansion element 210 is shown in a deflated configuration. The catheter 102 includes a guide tube 202, a coolant transfer lumen 204, and at least one coolant outtake region 206 each disposed in one or more flexible outer layers 208.

In some embodiments, the expansion element 108 includes a single member. In other embodiments, the expansion element 108 includes multiple members. For example, in at least some embodiments, the expansion element 108 includes an inner member 210 and an outer member 212 disposed over the inner member 210. FIGS. 1-3 show the expansion element 108 having two members. It will be understood that the expansion element 108 may, instead, only have a single member, or may have more than two members.

In at least some embodiments, a vacuum is maintained between the inner member 210 and the outer member 212. In at least some embodiments, a space between the inner member 210 and the outer member 212 is in fluid communication with the coolant-flow regulation system 114. In at least some embodiments, a proximal end of the expansion element 108 couples to the distal portion 104 of the catheter 104 such that a region within the inner member 210 is in fluid communication with the at least one coolant outtake region 206.

The expansion element 108 may be formed from any elastic or semi-elastic material, such as one or more thermoplastics (e.g., polyether block amide, or the like), or other plastics (e.g., nylon, urethane, or the like) that maintain elasticity over a wide range of temperatures, particularly at the temperature of the expanded coolant. In at least some embodiments, the expansion element 108 is semi-elastic, wherein the size of the expansion element 108 does not change in response to incremental changes in relative pressure that are below 5 psi (about 34.5×10³ Pa).

In at least some embodiments, the guide tube 202 is configured and arranged to receive a stiffening member (e.g., a stylet, or the like) to facilitate guiding of the catheter 102 to a target location within patient vasculature by providing additional rigidity to the catheter 102. The guide tube 202 may be formed from any flexible material (e.g., a thermoplastic, or the like) that maintains elasticity over a wide range of temperatures, particularly at the temperature of the expanded coolant.

The guide tube 202 defines a lumen that extends along a length of the catheter 102 from the proximal portion (106 in FIG. 1) of the catheter 102 to a position that is beyond the distal portion 104 of the catheter 102. In at least some embodiments, the guide tube 202 extends to a distal end of the expansion element 108 when the expansion element is in the deflated configuration. In at least some embodiments, the guide tube 202 extends to a distal end of the expansion element 108 when the expansion element is in an inflated configuration. In at least some embodiments, the distal end of the expansion element 108 is coupled to the guide tube 202.

The coolant outtake region 206 is configured and arranged to accommodate coolant exiting the expansion element 108. The coolant outtake region 206 extends along the length of the catheter 102 from the proximal portion (106 in FIG. 1) of the catheter 102 to the expansion element 108. In some embodiments, the coolant outtake region 206 includes one or more tubes that define one or more lumens. In other embodiments, the coolant outtake region 206 includes one or more open regions within the outer layer 208 of the catheter 102 and exterior to the guide tube 202 and the coolant transfer tube 204. In at least some embodiments, the coolant outtake region 206 is in fluid communication with the fluid-drawing source (114 in FIG. 1).

The coolant transfer tube 204 extends along the length of the catheter 102 from the proximal portion (106 in FIG. 1) of the catheter 102. The coolant transfer tube 204 defines a lumen. A proximal end of the lumen is coupled to the coolant source (112 in FIG. 1). A distal end of the lumen is in fluid communication with the expansion element 108. In at least some embodiments, the distal end of the coolant transfer tube 204 extends at least partially into the expansion element 108. In at least some embodiments, the coolant transfer tube 204 extends to a distal end of the expansion element 108 when the expansion element 108 is in a deflated configuration. In at least some embodiments, the coolant transfer tube 204 extends to the distal end of the expansion element 108 when the expansion element 108 is in an inflated configuration. In at least some embodiments, a distal end of the expansion element 108 is coupled to the coolant transfer tube 204. In at least some embodiments, the distal end of the guide tube 202 extends beyond the coolant transfer tube 204.

Typically, coolant input to the coolant transfer tube 204 (e.g., via the coolant source 112) is drawn to the expansion element 108 by a lower pressure in the expansion element 108. In at least some embodiments, the coolant source includes a pressurized container or pump. The coolant is output to the expansion element 108 as a sprayed liquid that vaporizes and atomizes as the liquid is output from the coolant transfer tube 204. In at least some embodiments, when the coolant enters the expansion element 108, the expansion element 108 absorbs heat, thereby reducing the temperature of the expansion element 108 to a temperature sufficiently low enough to ablate patient tissue upon contact.

The reduction in temperature of the expansion element 108 may be due to one or more of the Joule-Thomson effect or the latent heat of vaporization. The Joule-Thomson effect describes the cooling effect that comes about when a compressed non-ideal gas expands into a region of low pressure (e.g., within the expansion element 108). The latent heat of vaporization describes heat being released as a result of the phase change from a liquid to a gas (e.g., the liquefied coolant vaporizing upon entering the expansion element 108).

FIG. 2B is a schematic longitudinal cross-sectional view of one embodiment of the expansion element 108 in an inflated configuration. Directional arrows, such as arrow 216, show the flow of coolant from the coolant transfer tube 204 to an inner surface of the expansion element 108. The expanded gas dissipates down the catheter 102 along the coolant outtake region 206. In at least some embodiments, the fluid-drawing system (114 in FIG. 1) is used to draw the expanded, heated, and gaseous coolant along the coolant outtake region 206 from the expansion element 108 to a reduced pressure region. In at least some embodiments, the reduced pressure region is disposed along the coolant outtake region 206. In at least some embodiments, the reduced pressure region is in fluid communication with the coolant outtake region 206. In at least some embodiments, the reduced pressure region is external to the catheter 102. In at least some embodiments, the reduced pressure region is external to the cryoablation system 100. In at least some embodiments, the reduced pressure region is the ambient atmosphere.

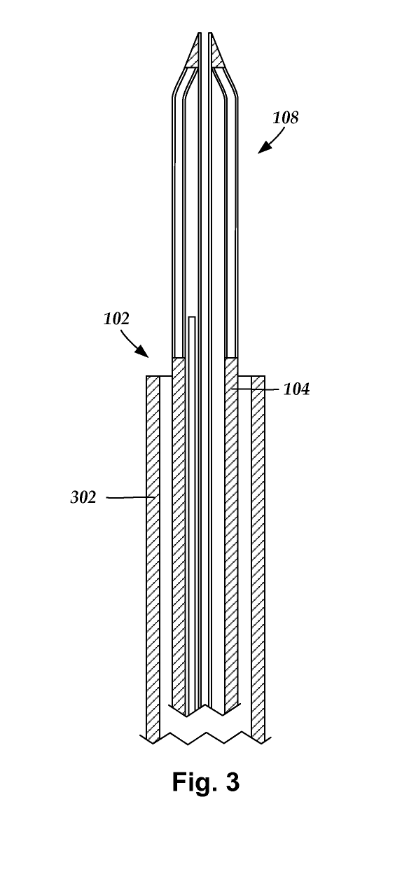

In at least some embodiments, a sheath may be used to facilitate guidance of the catheter through patient vasculature during insertion of the catheter. FIG. 3 is a schematic longitudinal cross-sectional view of one embodiment of the distal portion 104 of the catheter 102 disposed in a sheath 302. In at least some embodiments, the sheath 302 is steerable. Once the catheter 102 is positioned at the target ablation location, such as the left atrium of the patient's heart, adjacent to the ostia of the pulmonary veins, the sheath 302 can be removed. In at least some embodiments, the pressure within the expansion element 108 may be held constant for a period of time without reducing the temperature in the expansion element 108 to a temperature sufficient to ablate patient tissue by inputting coolant to the coolant transfer tube 204 at a rate that is sufficiently-low enough to prevent the creation of ablation temperatures in the expansion element 108. In at least some embodiments, such as when a pulmonary vein is the target ablation location, the expansion element 108 can be expanded to occlude the lumen of the pulmonary vein and to anchor the expansion element 108 at the target ablation location.

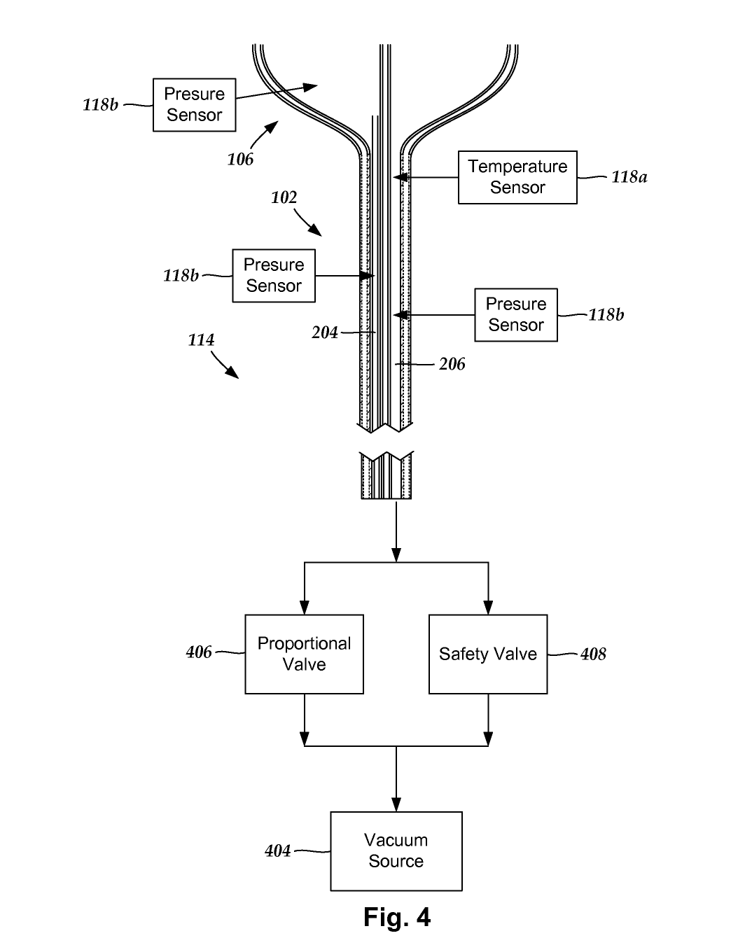

Turning now to FIG. 4, one concern during a cryoablation procedure is regulating the rate of coolant flow in the catheter (e.g., in the coolant transfer tube 204 and in the coolant outtake region 206) and in the expansion element 108. Another concern is maintaining a stable pressure in the expansion element 108, despite changes in the rate of coolant flow into the coolant transfer tube 204 during the cryoablation procedure. As mentioned above, a pressure differential exists along the catheter 102 during operation. The pressure differential drives the flow of coolant from the coolant source 112 to the region of reduced pressure, via the expansion element 108. The pressure differential is dependent on the properties of the gaseous coolant and the mass flow rate of the gaseous coolant. The pressure differential includes an exhaust pressure differential along the coolant outtake region 206, between the expansion element 108 and the reduced pressure region.

It is generally desirable to input coolant to the coolant transfer tube 204 at a rate that is below a thermal load of the cryoablation system 100. When coolant is delivered to the expansion element 108 at a rate that exceeds the thermal load of the cryoablation system 100, the liquid coolant input to the expansion element 108 may not fully vaporize. Thus, liquid coolant may collect in the expansion element 108 and may also flow into the coolant outtake region 206. Pooled liquid coolant in the expansion element 108 or the coolant outtake region 206 may cause adverse performance, or even failure, of the cryoablation system 100, and even potential danger to the patient. Reducing the rate of coolant flow into the coolant transfer tube 204 can reduce the pooling of liquid coolant in the expansion element 108. Reducing the rate of coolant flow into the coolant transfer tube 204, however, may cause a corresponding undesired partial deflation of the expansion element 108.

It is generally desirable to maintain a constant pressure in the expansion element 108 during a cryoablation procedure. For example, when the expansion element 108 is configured and arranged to expand, changes in coolant flow rate may cause variable pressure in the expansion element 108 which, in turn, may cause an undesired temporary partial deflation of the expansion element 108. As mentioned above, when the expansion element 108 is positioned at a target ablation location, mass flow of coolant into the coolant transfer tube 204 may occur for a period of time at a rate that is sufficiently slow enough to maintain a constant pressure in the expansion element 108 without a corresponding temperature reduction sufficient to ablation tissue upon contact. In some instances, a pressure reduction may occur in the expansion element 108 at some point between placement of the expansion element 108 at the target ablation location and the onset of ablation. For example, a pressure reduction may occur after the expansion element 108 is positioned, but before patient tissue adheres to the outer surface of the expansion element 108. In which case, the expansion element 108 may move more distally into the pulmonary vein. A subsequent expansion of the expansion element 108 may result in mechanical trauma to the pulmonary vein, such as stenosis, or one or more other adverse patient effects, such as onset of nerve palsy (e.g., phrenic nerve palsy).

The coolant-flow regulation system 114 regulates coolant flow within the catheter 102 and expansion element 108. In at least some embodiments, the coolant-flow regulation system 114 also regulates pressure within the expansion element 108. In at least some embodiments, the coolant-flow regulation system 114 regulates the rate of coolant flow by adjusting the exhaust pressure differential, thereby regulating the flow of coolant into the coolant transfer tube 204 by controlling the back pressure in the catheter 102.

Changes in the exhaust pressure differential from the coolant-flow regulation system cause corresponding changes in the rate of coolant flow into the coolant transfer tube 204. In at least some embodiments, changes in the exhaust pressure differential from the coolant-flow regulation system cause corresponding changes in the rate of coolant flow into the coolant transfer tube 204 such that the pressure within the expansion element 108 remains constant. In at least some embodiments, the exhaust pressure differential is controlled via at least one of adjusting impedance along the coolant outtake region 206. In at least some embodiments, the exhaust pressure differential is controlled via adjustment of one or more adjustable valves. In at least some embodiments, the exhaust pressure differential is controlled via the amount of vacuum generated by an adjustable vacuum source within the coolant-flow regulation system 114.

In at least some embodiments, the coolant-flow regulation system 114 includes one or more temperature sensors. In at least some embodiments, the coolant-flow regulation system 114 includes one or more pressure sensors. In at least some embodiments, sensed changes in pressure or temperature at various locations along the catheter 102 and expansion element 108 can be used to initiate changes in the exhaust pressure differential. For example, in at least some embodiments, when the coolant-flow regulation system 114 senses liquid coolant (e.g., via the temperature sensor 118a of FIG. 4) in a distal end of the coolant outtake region 206, the rate of coolant flow along the coolant outtake region 206 is adjusted, thereby causing a corresponding change in the rate of coolant flow input to the coolant transfer tube 204.

As another example, in at least some embodiments, when the coolant-flow regulation system 114 senses decreased pressure in one or more of the coolant transfer tube 204, the expansion element 108, or the coolant outtake region 206, the rate of coolant flow along the coolant outtake region 206 is adjusted, thereby causing a corresponding change in the rate of coolant flow input to the coolant transfer tube 204.

As discussed above, in at least some embodiments the coolant-flow regulation system 114 causes changes in the rate of coolant flow along the coolant outtake region 206 by causing at least one of changes in impedance along the coolant outtake region 206 or adjusting the parameters of the vacuum source along the coolant outtake region 206. In at least some embodiments, the coolant-flow regulation system 114 includes one or more valves for adjusting impedance along the coolant outtake region 206. In at least some embodiments, impedance along the coolant outtake region 206 can be regulated, at least in part, by opening and closing the one or more valves. In at least some embodiments, the one or more valves are disposed on the coolant outtake region 206. In at least some embodiments, the one or more valves are disposed in fluid communication with the coolant outtake region 206. In at least some embodiments, the vacuum source is disposed on the coolant outtake region 206. In at least some embodiments, the vacuum source is disposed in fluid communication with the coolant outtake region 206.

In at least some embodiments, changes in at least one of exhaust impedance or vacuum source parameters are used to cause corresponding changes in the rate of coolant flow along the coolant outtake region 206. For example, the pressure in the expansion element 108 can be increased by either decreasing the vacuum level or increasing exhaust impedance (or both). In response, the coolant-flow regulation system 114 adjusts the rate of coolant flow into the coolant transfer tube 204. In at least some embodiments, when the coolant-flow regulation system 114 adjusts the rate of coolant flow into the coolant transfer tube 204, the adjustment to the rate of coolant flow is performed such that a stable inflated pressure is maintained in the expansion element 108.

In another example, when the catheter 102 is positioned against a target ablation region (e.g., against the ostium of the pulmonary vein, or the like), the coolant-flow regulation system 114 can increase impedance (or decrease the vacuum) along the coolant outtake region 206 to maintain a constant pressure in the expansion element 108 without reducing temperature in the expansion element 108 to a temperature sufficient for ablating patient tissue. In at least some embodiments, the coolant-flow regulation system 114 can then decrease impedance (or increase the vacuum) along the coolant outtake region 206 while increasing the flow of coolant along the coolant transfer tube 204, thereby reducing the average temperature within the expansion element 108 while also maintaining a constant pressure in the expansion element 108.

In other words, the increased mass flow provides additional cooling power. Cooling power is equal to heat transfer rate which, in turn, is equal to the enthalpy change multiplied by the mass flow rate. Temperature in the expansion element 108 is non-uniform under high heat transfer rates. Under low flow conditions, there may be insufficient power to offset the heat transfer rate into the expansion element 108. Thus, the temperature may remain high. Increasing flow rate while maintaining (or at least controlling) the pressure in the expansion element 108 may cause the average temperature within the expansion element 108 to drop.

In at least some embodiments, this process can be performed in reverse, after the expansion element 108 is at a temperature sufficient to ablate patient tissue upon contact (e.g., after a cryoablation procedure or a portion of a cryoablation procedure). In at least some embodiments, the process can also be performed during an ablation cessation during an ablation procedure (e.g., when the rate of coolant flow into the coolant transfer tube 204 exceeds the thermal load of the cryoablation system 100, or the like).

FIG. 4 is a schematic view of one embodiment of a coolant-flow regulation system for regulating exhaust pressure differential within the cryoablation system 100. The coolant-flow regulation system 114 includes a vacuum pump 404 and one or more valves 406 and 408. In at least some embodiments, the valve 406 is an adjustable valve, such as a proportional valve. In at least some embodiments, the valve 408 is a safety valve, such as a low side valve. In at least some embodiments, the valves 406, 408 are in parallel to one another. In at least some embodiments, the valves 406, 408 are in series to one another. In at least some embodiments, the valves 406, 408 are in parallel with the vacuum source 404. In at least some embodiments, the valves 406, 408 are in series with the vacuum source 404.

In at least some embodiments, exhaust impedance is controllable by adjusting the valve 406. In at least some embodiments, the safety valve 408 remains closed during normal operation. In at least some embodiments, the safety valve 408 opens during emergency situations when an immediate decrease in impedance along the coolant outtake region 206 is desired. In at least some embodiments, at least one of the valves 406, 408 opens to a region exterior to the catheter 102. In at least some embodiments, at least one of the valves 406, 408 opens to a region exterior to the patient during a cryoablation system. In at least some embodiments, at least one of the valves 406, 408 opens to the ambient atmosphere. It will be understood that one or more other valves may be employed in addition to, or in lieu of, the one or more valves 406, 408.

In FIG. 4, the coolant-flow regulation system 114 includes one or more temperature sensors 118a. In at least some embodiments, at least one of the temperature sensors 118a is disposed at a distal end of the coolant outtake region 206. The coolant-flow regulation system 114 shown in FIG. 4 also includes one or more pressure sensors 118b. In at least some embodiments, at least one of the pressure sensors 118b is disposed in the coolant transfer tube 204. In at least some embodiments, at least one of the pressure sensors 118b is disposed in the expansion element 108. In at least some embodiments, at least one of the pressure sensors 118b is disposed in the coolant outtake region 206.

Accordingly, in at least some embodiments the coolant flow rate through the catheter 102 may be changed, as needed, during a cryoablation procedure while maintaining a stable inflated pressure in the expansion element 108 through selective application of at least one of impedance change or vacuum level adjustment within the coolant outtake region 206. In at least some embodiments, the exhaust pressure differential is controlled by monitoring the pressure (e.g., using one or more pressure sensors 118b) or temperature (e.g., using one or more temperature sensors 118a) and adjusting the exhaust pressure differential. The resulting changes to the back pressure cause corresponding changes to the rate of coolant flow into the coolant transfer tube 204. In at least some embodiments, the induced changes to the rate of coolant flow into the coolant transfer tube 204 maintain a constant pressure in the expansion element 108.

It will be understood that impedance along the coolant outtake region 206 can be adjusted by implementing any method of restricting flow of coolant along the coolant outtake region 206 in addition to, or in lieu of, the one or more valves 406, 408. It will also be understood that one or more components (e.g., one or more of the valves 406, 408, the vacuum source 404, or one or more of the sensors 118a, 118b) of the coolant-flow control system 114 can be controlled either electronically via one or more processors (e.g., disposed in the control module, or disposed in a stand-alone unit separate from the cryoablation system 100), or manually by a medical practitioner, or both.

In at least some embodiments, the expansion element 108 is inflated to an absolute pressure that is no more than 2 atm (about 2×10⁵ Pa). In at least some embodiments, the expansion element 108 is inflated to an absolute pressure that is no more than 3 atm (about 3×10⁵ Pa). In at least some embodiments, the expansion element 108 is inflated to an absolute pressure that is no more than 4 atm (about 4×10⁵ Pa). In at least some embodiments, the expansion element 108 is inflated to an absolute pressure that is no more than 5 atm (about 5×10⁵ Pa). In at least some embodiments, the expansion element 108 is inflated to an absolute pressure that is no more than 6 atm (about 6×10⁵ Pa). In at least some embodiments, the expansion element 108 is inflated to an absolute pressure that is no more than 7 atm (about 7×10⁵ Pa). In at least some embodiments, the expansion element 108 is inflated to an absolute pressure that is no more than 8 atm (about 8×10⁵ Pa). In at least some embodiments, the expansion element 108 is inflated to an absolute pressure that is no more than 9 atm (about 9×10⁵ Pa).

In at least some embodiments, the temperature of the expansion element 108 is reduced to a temperature that is no greater than −20° C. In at least some embodiments, the temperature of the expansion element 108 is reduced to a temperature that is no greater than −40° C. In at least some embodiments, the temperature of the expansion element 108 is reduced to a temperature that is no greater than −60° C. In at least some embodiments, the temperature of the expansion element 108 is reduced to a temperature that is no greater than −80° C. In at least some embodiments, the temperature of the expansion element 108 is reduced to a temperature that is no greater than −100° C.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A cryoablation system comprising:
a catheter having a distal portion, a proximal portion, and a length, the catheter configured and arranged for insertion into patient vasculature, the catheter comprising
a coolant transfer tube extending along the catheter, the coolant transfer tube defining a lumen configured and arranged to receive and transfer coolant from a coolant source, and
at least one coolant outtake region extending along the catheter; and
an expansion element coupled to the distal portion of the catheter, the expansion element in fluid communication with the coolant transfer tube and the at least one coolant outtake region, wherein the coolant outtake region is configured and arranged to receive and transfer coolant from the expansion element to a reduced pressure region;
a coolant-flow regulation system at least partially disposed in the catheter, the coolant-flow regulation system configured and arranged for regulating a rate of coolant flow within the cryoablation system by providing an adjustable pressure differential along the at least one coolant outtake region; and
a control module coupled to the catheter, the control module comprising a processor for controlling the coolant-flow regulation system.

2. The cryoablation system of claim 1, wherein the adjustable pressure differential along the at least one coolant outtake region is provided by a vacuum source in fluid communication with the at least one coolant outtake region.

3. The cryoablation system of claim 1, wherein the adjustable pressure differential along the at least one coolant outtake region is provided by at least one adjustable valve disposed along the at least one coolant outtake region, the at least one adjustable valve configured and arranged to adjust impedance along the coolant outtake region.

4. The cryoablation system of claim 1, wherein the coolant-flow regulation system comprises at least one safety valve disposed along the at least one coolant outtake region.

5. The cryoablation system of claim 1, wherein the control module further comprises a coolant source coupled to the coolant transfer tube.

6. The cryoablation system of claim 1, wherein when the expansion element is in an expanded position, the coolant-flow regulation system is configured and arranged for regulating the rate of coolant flow such that the pressure within the expansion element remains constant.

7. The cryoablation system of claim 1, wherein the coolant-flow regulation system comprises at least one pressure sensor disposed in the coolant transfer tube.

8. The cryoablation system of claim 1, wherein the coolant-flow regulation system comprises at least one pressure sensor disposed in the at least one coolant outtake region.

9. The cryoablation system of claim 1, wherein the coolant-flow regulation system comprises at least one pressure sensor disposed in the expansion element.

10. The cryoablation system of claim 1, wherein the coolant-flow regulation system comprises at least one temperature sensor disposed at a distal end of the at least one coolant outtake region.

11. A method for cryoablating patient tissue, the method comprising:
inserting a catheter and an expansion element in patient vasculature, the catheter having a distal portion, a proximal portion, and a length, the catheter comprising a coolant transfer tube extending along the catheter and at least one coolant outtake region, wherein the expansion element is disposed at the distal portion of the catheter;
guiding the expansion element in proximity to a target ablation location within the patient;
drawing coolant from a coolant source such that coolant flows along the coolant transfer tube and enters into the expansion element, thereby reducing the temperature of the expansion element to a temperature sufficiently low enough to ablate patient tissue at the target ablation location upon contact;
adjusting a pressure differential along the at least one coolant outtake region using a coolant-flow regulation system at least partially disposed in the catheter;
adjusting a rate of coolant flow into the coolant transfer tube in response to the adjustment of the pressure differential along the at least one coolant outtake region; and
contacting patient tissue at the target ablation location with the expansion element for a time period adequate to ablate tissue contacting the expansion element.

12. The method of claim 11, wherein adjusting the pressure differential along the at least one coolant outtake region using the coolant-flow regulation system comprises adjusting one or more parameters of a vacuum generated by a vacuum source disposed in fluid communication with the at least one coolant outtake region.

13. The method of claim 11, wherein adjusting the pressure differential along the at least one coolant outtake region using the coolant-flow regulation system comprises adjusting one or more adjustable valves within the at least one coolant outtake region.

14. The method of claim 11, further comprising monitoring pressure within the expansion element using at least one pressure sensor.

15. The method of claim 11, further comprising monitoring pressure within the coolant transfer tube using at least one pressure sensor.

16. The method of claim 11, further comprising monitoring pressure within the at least one coolant outtake region using at least one pressure sensor.

17. The method of claim 11, further comprising monitoring pressure within a distal end of the at least one coolant outtake region using at least one temperature sensor.

18. The method of claim 17, further comprising detecting excessive saturated liquid in the expansion element by sensing a temperature reduction in the distal end of the at least one coolant outtake region using the at least one temperature sensor.

19. The method of claim 18, further comprising opening a safety valve in the at least one coolant outtake region in response to the detection of saturated liquid in the distal end of the at least one coolant outtake region.

20. The method of claim 11, wherein adjusting the rate of coolant flow into the coolant transfer tube in response to the adjustment of the pressure differential along the at least one coolant outtake region comprises maintaining a constant pressure within the expansion element.
